(19) United States
(12) Patent Application Publication
(10) Pub. No.: US 2021/0371886 A1
(43) Pub. Date: Dec. 2, 2021

(54) PROCESS FOR CONTROLLING A FERMENTATION PROCESS

(71) Applicant: FermentationExperts A/S, Bække (DK)

(72) Inventor: Søren Kjærulff, Copenhagen (DK)

(21) Appl. No.: 17/277,585

(22) PCT Filed: Sep. 18, 2019
(86) PCT No.: PCT/EP2019/074992

§ 371 Date: Mar. 18, 2021

(30) Foreign Application Priority Data
Sep. 19, 2018 (DK) PA 201800610

(51) Int. Cl. C12P 7/56; C12P 13/08; C12P 7/40

(52) U.S. Cl. CPC C12P 7/56; C12P 7/40; C12P 13/08

(57) ABSTRACT

A process controlling development of at least two predetermined metabolites in a fermented product comprising at least one plant material and/or at least one seaweed material, the process comprising the steps of: (i) Determining the at least 2 predetermined metabolites to be developed in the fermented product; (ii) Based on the predetermined metabolites determined in step (i) selecting at least one plant material and/or the at least one seaweed material; (iii) Based on the predetermined metabolites determined in step (i) selecting at least one fermenting organism; (iv) Mixing the at least one plant material and/or the at least one seaweed material selected in step (ii) with the at least one fermenting organism selected in step (iii) in a fermentation reactor providing a fermentation mixture; (v) Allowing the fermentation mixture to ferment under fermentation conditions favoring development of the at least two predetermined metabolites; whereby the fermented product is provided.

### TECHNICAL FIELD OF THE INVENTION

The present invention relates to a process for controlling a fermentation process and for providing a quality control of a fermentation product. In particular the present invention relates to a process for controlling a fermentation process when fermenting at least one plant material and/or at least one seaweed material in order to ensure development of desired and/or predefined components.

### BACKGROUND OF THE INVENTION

Fermentation is a metabolic process where a fermenting organism consumes a carbohydrate source and produces various metabolites.

When fermenting plant materials and seaweed materials, the materials act as carbohydrate source for the fermenting organism and the materials are degraded resulting in development of various metabolites. During the fermentation process the digestibility of the materials, which normally may be substantially indigestible materials, may change and may additionally provide a prebiotic effect to the human or animal consuming the fermented product.

The plant materials and seaweed materials may be provided in many different qualities and many different variations of the same plant material or seaweed material depending on the geographic location, the weather, the time of harvesting, the handling after harvesting and before fermentation; the storage conditions etc.

These variations may affect the fermentation process and create significant difficulties in providing uniform fermented products from the fermented plant materials and/or the fermented seaweed materials. Hence, there is a need in the industry, in particular when working with large or industrial scale, for a fermentation process resulting in uniform fermented products also comprising plant materials or seaweed materials,

Traditionally, the uniform products are provided from fermentation products which after fermentation is subjected to isolation or extraction in order to provide uniform active components or uniform active fractions, however, the same tendency to provide uniform and special targeted fermented products comprising the originally present plant materials and/or the originally present seaweed materials, is not custom as the fermented products may vary significantly in quality and constituents.

Hence, even being the same plant material or being the same seaweed material large differences in the material as well as in the traditional fermented products may be observed.

Thus, there is a need in the industry to provide a process capable of ensuring uniform fermentation products and to ensure fermentation products having the desired constituents.

Hence, an improved process for fermenting plant materials and/or seaweed materials would be advantageous, and in particular a more efficient, uniform, adaptable and/or reliable process for fermenting plant materials and/or seaweed materials would be advantageous.

### SUMMARY OF THE INVENTION

Thus, an object of the present invention relates to an improved process for controlling the fermentation of a plant material and/or a seaweed material.

In particular, it is an object of the present invention to provide a more efficient, uniform, adaptable and/or reliable process for fermenting plant materials and/or seaweed materials that solves the above-mentioned problems of the prior art with diversity between batches.

Thus, one aspect of the invention relates to a process for controlling the development of at least 2 predetermined metabolites in a fermented product comprising at least one plant material and/or at least one seaweed material, wherein the process comprising the steps of:
(i) Determining the at least 2 predetermined metabolites to be developed in the fermented product;
(ii) Based on the predetermined metabolites determined in step (i) at least one plant material and/or the at least one seaweed material is selected;
(iii) Based on the predetermined metabolites determined in step (i) at least one fermenting organism is selected;
(iv) Mixing the at least one plant material and/or the at least one seaweed material selected in step (ii) with the at least one fermenting organism selected in step (iii) in a fermentation reactor providing a fermentation mixture;
(v) Allowing the fermentation mixture to ferment under fermentation conditions favouring the development of the at least 2 predetermined metabolites;
whereby the fermented product is provided.

Another aspect of the present invention relates to a fermented product comprising:
(a) at least one plant material and/or at least one seaweed material;
(b) at least one fermenting organism;
(c) at least 2 metabolites, wherein the at least 2 metabolites are selected from an amino acid; a fatty acid; a bioactive phenol; a vitamin; an acid; a purine compound; a carbohydrate compound; a flavonoid compound; or a bacterial biomarker.

Yet another aspect of the present invention relates to the use of at least 2 metabolites for controlling a fermentation process for providing a fermented product according to the present invention.

Still another aspect of the present invention relates to the use of a library of metabolites for controlling a fermentation process for providing a fermented product according to the present invention.

An even further aspect of the present invention relates to a fermented product comprising a library of metabolites selected from an amino acid; a fatty acid; a bioactive phenol; a vitamin; an acid; a purine compound; a carbohydrate compound; a flavonoid compound; or a bacterial biomarker.

### BRIEF DESCRIPTION OF THE FIGURES

FIG. 1 shows a fingerprint comprising numerous of metabolites of a fermented product according to the present invention using the lactic acid bacteria, Pediococcus acidilactici DSM 16243, Pediococcus pentosaceus DSM 12834 and Lactobacillus plantarum DSM 12837. The fermented product comprising rape material, mustard material and seaweed material.

FIG. 2 shows the development and amount of a specific metabolite, benzoic acid, in a fermented product comprising rape material, mustard material and seaweed material using the lactic acid bacteria, Pediococcus acidilactici DSM 16243, Pediococcus pentosaceus DSM 12834 and Lactobacillus plantarum DSM 12837. As illustrated in FIG. 2, then benzoic acid may only be formed when fermenting seaweed materials.

FIG. 3 shows the development and amount another specific metabolite, lysine, in a fermented product obtained from fermenting rape material, mustard material and seaweed material using the lactic acid bacteria, Pediococcus acidilactici DSM 16243, Pediococcus pentosaceus DSM 12834 and Lactobacillus plantarum DSM 12837.

The present invention will now be described in more detail in the following.

### DETAILED DESCRIPTION OF THE INVENTION

When working with natural materials like plant materials and seaweed many changes may occur in the same plant material or seaweed material depending on the geographic location, the weather, the time of harvesting, the handling after harvesting and before fermentation; the storage conditions etc. Hence, even being the same plant material or being the same seaweed material large differences in the material may be observed. Thus, in order to ensure a uniform fermentation product and to ensure a fermentation product having the desired constituents the fermentation process may be controlled, in particular in terms of the development of specific metabolites produced during the fermentation process.

By controlling the process according to the present invention, it may be possible to provide specific fermentation product with specific functional features and/or improve certain functional features compared with the prior art.

Hence, a preferred embodiment of the present invention relates to a process for controlling the development of at least 2 predetermined metabolites in a fermented product comprising at least one plant material and/or at least one seaweed material, wherein the process comprising the steps of:
(i) Determining the at least 2 predetermined metabolites to be developed in the fermented product;
(ii) Based on the predetermined metabolites determined in step (i) at least one plant material and/or the at least one seaweed material is selected;
(iii) Based on the predetermined metabolites determined in step (i) at least one fermenting organism is selected;
(iv) Mixing the at least one plant material and/or the at least one seaweed material selected in step (ii) with the at least one fermenting organism selected in step (iii) in a fermentation reactor providing a fermentation mixture;
(v) Allowing the fermentation mixture to ferment under fermentation conditions favouring the development of the at least 2 predetermined metabolites;
whereby the fermented product is provided.

In the present context the term “predetermined metabolite” relates to the development of metabolite(s) selected prior to the start of the fermentation, in order to either increase the development of said metabolite(s) or decrease or suppress the development of said metabolite(s). It may be that some predetermined metabolites are to have an increased metabolite(s) as well as a metabolite which is to be suppressed.

In an embodiment of the present invention the at least 3 predetermined metabolites are determined to be developed in the fermentation process, such as at least 4 predetermined metabolites, e.g. at least 5 predetermined metabolites, such as at least 6 predetermined metabolites, e.g. at least 8 predetermined metabolites, such as at least 10 predetermined metabolites, e.g. at least 15 predetermined metabolites, such as at least 20 predetermined metabolites, e.g. at least 25 predetermined metabolites, such as at least 30 predetermined metabolites, e.g. at least 35 predetermined metabolites, such as at least 40 predetermined metabolites, e.g. at least 45 predetermined metabolites, such as at least 50 predetermined metabolites.

In a further embodiment of the present invention, the at least 2 metabolites of the present invention represent a fingerprint of the fermented product. In an embodiment of the present invention the fingerprint provides an indication of (i) the presence of the at least 2 predetermined metabolites and (ii) the content of the at least 2 predetermined metabolites, present in the fermented product. The fingerprint may be illustrated in a mass spec or in a simple list.

Some variation between various fingerprints comprising the same predetermined metabolites may occur, due to the analysis and the variation may be increased if the number of predetermined metabolites is increased. In the event a variation between various fingerprints is observed, the ratio between the at least 2 predetermined metabolites may be the same, or substantially the same.

In the present context the term “development” relates to the transformation of the at least one plant material and/or the at least one seaweed material into metabolites. The development may either be as an increased formation of a metabolite or it may be as a decreased or a suppressed formation of a metabolite

During fermentation of at least one plant material and/or the at least one seaweed material multiple metabolites may be formed.

Fermentation products according to the present invention relates to a complex mixture of components, including components originally originating from the at least one plant material and/or the at least one seaweed material, and components produced during the fermentation process. The at least 2 metabolites according to the present invention are preferably, produced during the fermentation process.

In an embodiment of the present invention a library of metabolites may be provided, optionally in combination with an indication of the concentration of the various metabolites, an may act as a fingerprint of the fermented product.

This fingerprint of the fermented product may be compared with a previous fingerprint in order to evaluate the quality of the fermented product and/or to evaluate the uniformity of the fermented products provided.

In an embodiment of the present invention a library of metabolites may be identified in the fermented product.

In the present context the term “identified metabolites” relates to both known and unknown metabolites identified by e.g. GC-MS analysis (Gas Chromatography-Mass Spectrometry analysis) or HILIC analysis (Hydrophilic interaction chromatography analysis). Preferably, the known compound may be characterised and named.

In an embodiment of the present invention a library of metabolites of the fermented product and/or the fermentation mixture may be compared to a library of metabolites of a similar previous fermented product.

In the present context the term “library of metabolites” relates to range of metabolites. Preferably the library of metabolites comprises at last 20 metabolites, such as at least 30 metabolites, e.g. at least 40 metabolites, such as at least 50 metabolites, e.g. at least 60 metabolites, such as at least 70 metabolites, e.g. at least 80 metabolites, such as at least 80 metabolites, e.g. at least 90 metabolites, such as at least 100 metabolites, e.g. at least 250 metabolites, such as at least 500 metabolites.

In an embodiment of the present invention the at least 2 metabolites may be selected from an amino acid; a fatty acid; a bioactive phenol; a vitamin; an acid; a purine compound; a carbohydrate compound; a flavonoid compound; or a bacterial biomarker.

The amino acid may be selected from the group consisting of alanine (ala); arginine (arg); asparagine (asn); aspartic acid (asp); cysteine (cys); glutamine (gin); glutamic acid (glu); glycine (gly); histidine (his); isoleucine (ile); leucine (leu); lysine (lys); methionine (met); phenylalanine (phe); proline (pro); serine (ser); threonine (thr); tryptophan (trp); tyrosine (tyr); and valine (val).

Preferably, the amino acid may be selected from the group consisting of arginine (arg); asparagine (asn); aspartic acid (asp); glutamine (gin); glutamic acid (glu); isoleucine (ile); lysine (lys); phenylalanine (phe); proline (pro); tryptophan (trp); and tyrosine (tyr).

In an embodiment of the present invention the amino acid may be an essential amino acid. Preferably, the essential amino acid may be selected from the group consisting of histidine (his); isoleucine (ile); leucine (leu); lysine (lys); methionine (met); phenylalanine (phe); threonine (thr); tryptophan (trp); and valine (val). In particular, the essential amino acid is selected from the group consisting of isoleucine (ile); leucine (leu); lysine (lys); phenylalanine (phe); and tryptophan (trp).

In a further embodiment of the present invention the fatty acid may be a polyunsaturated fatty acid; an unsaturated fatty acid, or a short-chain fatty acid.

Preferably, the polyunsaturated fatty acids may preferably be selected from methylene-interrupted polyenes; Omega-3, Omega-6 and Omega-9, Conjugated fatty acids or other polyunsaturated fatty acids selected from linolenic acid (C18:2), γ-linolenic acid (C18:3); α-linolenic acid (C18:3); octadecatetraenoic acid (18:4); Eicosatetraenoic acid (C20:4); Eicosapentaenoic acid (C20:5); or Pinolenic acid

The unsaturated fatty acid may preferably be selected from oleic acid (C18:1); enoic acid (C18:1); 10-undecenoic acid (C10:1); hydroxystearic acid (C10:1); 9-hexadecanoic acid (C16:1).

The short-chain fatty acid may preferably be selected from Formic acid; Acetic acid; Propionic acid; Butyric acid, Isobutyric acid, Valeric acid and Isovaleric acid.

In an embodiment of the present invention the bioactive phenol is protocatechuic, p-hydroxybenzoic, 2,3-dihydroxybenzoic, chlorogenic, vanillic, caffeic, p-coumaric, salicylic acid, flavonoids.

In a further embodiment of the present invention the acid may be an organic or an inorganic acid.

The inorganic acid may preferably be taurine.

The organic acid may be selected from a nitrogenic organic acid; an aromatic organic acid; an aliphatic organic acid; or a dicarboxylic acid.

Preferably, the nitrogenic organic acid may be creatine; carnitine or derivatives hereof, such as acetyl-carnitine, burtyryl-carnitine, deoxy-carnitine, isovaleryl-carnitine, and propionyl-carnitine.

The aromatic organic acid may preferably be benzoic acid; nicotinic acid; or derivatives hereof.

In a preferred embodiment of the present invention one of the at least 2 metabolites may be benzoic acid.

The aliphatic organic acid may preferably be selected from lactic acid; acetic acid; aconitic acid; isocitric acid; or citric acid.

The dicarboxylic acid may preferably be selected from 2-oxoglutaric acid; malic acid; pyruvic acid; fumaric acid; succinic acid; or malonic acid.

In an embodiment of the present invention the purine compound may be selected from an adenine compound; an adenosine compound; a cytidine compound; a cytosine compound; a guanine compound; a guanosine compound; a hypoxanthine compound, an inosine compound; or a xanthine compound.

In a further embodiment of the present invention the carbohydrate compound may be selected from a fructose 6-phosphate; a glucose; a glucose 6-phosphate; or a myo-inositol.

In yet an embodiment of the present invention the flavonoid compound may be a kaempferol compound, such as kaempferol 3-O-sophoroside.

When controlling the fermentation process as described herein according to the present invention, it may be possible to design the fermentation process and the materials to be fermented based on the intended use of the fermented product. In this way it may be possible to improve the activity and/or the specificity of a fermented product.

In the present context the term “improved activity” relates to health promoting activities of the consumer, it may be antimicrobial activity to the fermented product or to the consumer; improving digestibility of the fermented product; improving taste and/or smell of the product.

In the context of the present invention, the term “specificity” relates to the ability of the fermented product to provide specific activities. In an embodiment of the present invention the activities may be health promoting activities of the consumer, it may be antimicrobial activity to the fermented product or to the consumer; improving digestibility of the fermented product; improving taste and/or smell of the product.

In an embodiment of the present invention the at least 2 predetermined metabolites does not include HDMPPA (3-(4′-hydroxyl-3′,5′-dimethoxyphenyl)propionic acid).

The inventors of the present invention surprisingly found that four parameters may show to be particular important in controlling the fermentation process, these parameters may include:
(I) the type of seaweed material;
(II) the type of plant material;
(III) the fermenting organism(s) selected; and/or
(IV) the fermentation process.

By regulating one or more of these 4 parameters it is possible manipulate development of certain metabolites of interest in order to control the fermentation process. In this way it may be possible to provide uniform, or substantially uniform, products.

The first parameter which may be found to be particular important for controlling the development of a metabolite during a fermentation process may be the type of seaweed material used.

The term “at least one fermented seaweed material” implies that different fermented seaweed materials may be used. In an embodiment of the present invention the fermented product comprises at least one seaweed material, such as at least two seaweed materials, e.g. at least three seaweed materials, such as at least four seaweed materials.

In an embodiment of the present invention, wherein the at least one fermented seaweed material may be a unicellular alga or a multicellular macroalgae.

In a further embodiment of the present invention, the multicellular macroalgae may be selected from brown macroalgae, red macroalgae, and/or green macroalgae.

In another embodiment of the present invention, the brown macroalgae may be selected from one or more of kelps, Saccharina latissima (Laminaria saccharina), Laminaria digitate, Ascophyllum nodosum, Laminaria hyperborean, or a mixture hereof.

The second parameter which may be found to be particular important for controlling the development of a metabolite during a fermentation process may be the type of plant material used.

In the present context, the term “plant material” relates to a material capable of performing photosynthesis.

The term “at least one fermented plant material” implies that different plant materials may be used. In an embodiment of the present invention the fermented product comprises at least one fermented plant material, such as at least two fermented plant materials, e.g. at least three fermented plant materials, such as at least four fermented plant materials.

When the fermented product comprises two or more fermented plant materials, the fermented plant materials may be of different origin.

In an embodiment of the present invention, the fermented plant material may be selected from at least one proteinaceous plant material. The proteinaceous plant material may be a vegetable plant material, preferably, the vegetable plant material may be selected from eudicot plants, angiosperm plants, and/or rosid plants.

Preferably the proteinaceous plant material or the vegetable plant material may be selected from Brassicale plants.

In an embodiment of the present invention the Brassicale plants is selected from the Brassicaceae family or the Cruciferae family.

In a further embodiment of the present invention, the Brassicaceae family or the Cruciferae family may be selected from at least one of a Brassica genus; Camelina genus; sun flower; palm; soya, field beans, lupins; or a combination hereof. Preferably, at least one Brassica genus may be selected from one or more species such as Brassica napus; Brassica oleracea; Brassica campestris; Brassica nigra; Sinapis alba (Brassica alba); Brassica juncea; Brassica rapa or mixtures hereof.

In yet an embodiment of the present invention, the at least one Brassica genus may be selected from the group consisting of: including rape, rapeseed, canola, cabbage, broccoli, cauliflower, kale, Brussels sprouts, collard greens, savoy, kohlrabi, gai Ian, white mustard, Indian mustard, Chinese mustard, and black mustard seed powder.

The fermented product may comprise a combination of at least one fermented seaweed material as defined herein, and at least one fermented plant material as defined herein.

In the present context, the term “fermented” relates to a material (at least one plant material, at least one seaweed material or a combination of at least one plant material and at least one seaweed material) relates to a controlled metabolic process of the material(s) by adding a predetermined amount of fermenting microorganisms to the material allowing the microorganism and the material(s) to interact breaking down the material.

The third parameter which may be found to be particular important for controlling the development of a metabolite during a fermentation process may be the type of fermenting organism used.

In an embodiment of the present invention the at least one fermenting organism may be one or more health-enhancing microorganism.

The fermented product and/or the fermentation mixture comprises one or more health-enhancing microorganism, preferably the health-enhancing microorganism may be a health-enhancing yeast and/or a health-enhancing bacterium, even more preferably the health-enhancing microorganism may be a health-enhancing bacterium.

The health-enhancing bacteria may comprise one or more probiotics. The one or more probiotics and/or the one or more health-enhancing microorganism may comprise at least one lactic acid bacterial strain.

In the context of the present invention, the term “probiotic” relates to live microorganisms that when administered in adequate amounts, confer a health benefit on the host.

In a preferred embodiment of the present invention, the at least one lactic acid bacterial strain may be selected from the group consisting of the genus Enterococcus, Lactobacillus, Pediococcus, Lactococcus, or Bifidobacterium or combinations thereof.

In a further embodiment of the present invention, the one or more lactic acid bacterial stain(s) may be selected from the group consisting of Pediococcus pentosaceus; Pendiococcus acidilactici; Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentosus, Lactobacillus vaginalis, Lactobacillus xylosus and a combination thereof.

In an embodiment of the present invention, the health-enhancing microorganism may be the main microorganism present in the fermented product. Preferably, the main microorganism is a lactic acid bacterium. Even more preferably the main microorganism may be selected from the groups consisting of Pediococcus pentosaceus; Pendiococcus acidilactici, Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentoses, Lactobacillus vaginalis, and Lactobacillus xylosus; preferably, the main lactic acid bacteria present in the composition is Lactobacillus plantarum.

In the context of the present invention, the term “main microorganism” relates to the microorganism present in highest amount, determined on a weight/weight ratio relative to the total number of microorganism present.

During fermentation, a group of microorganisms may be used for fermenting the plant material to provide a co-fermentation. The co-fermentation may be a mixture of different microorganisms (such as a mixture of yeasts, fungus, and/or bacteria) or a mixture of different bacteria. Preferably the co-fermentation comprises a mixture of different bacterial strains. In an embodiment of the present invention the fermenting mixture and/or the fermented product comprises one or more bacterial strains, e.g. two or more bacterial strains, such as three or more bacterial strains, e.g. four or more bacterial strains, such as 7 or more bacterial strains, e.g. 10 or more bacterial strains, such as 15 or more bacterial strains, e.g. 20 or more bacterial strains, such as 25 or more bacterial strains, e.g. 30 or more bacterial strains, such as 35 or more bacterial strains, e.g. 40 or more bacterial strains. Preferably, the bacterial strains may be one or more lactic acid bacterial strains.

In a further embodiment of the present invention, the one or more lactic acid bacteria stain(s) may be selected from the group consisting of one or more of Pediococcus pentosaceus (DSM 12834); Pendiococcus acidilactici (DSM 16243); Lactobacillus plantarum (DSM 12837); Enterococcus faecium (NCIMB 30122), Lactobacillus rhamnosus (NCIMB 30121), Pediococcus pentosaceus HTS (LMG P-22549), Pendiococcus acidilactici (NCIMB 30086) and/or Lactobacillus plantarum LSI (NCIMB 30083). Preferably, the one or more lactic acid bacteria stain(s) may be selected from the group consisting of one or more of Pediococcus pentosaceus (DSM 12834); Pendiococcus acidilactici (DSM 16243); Lactobacillus plantarum (DSM 12837).

The fermented product may have a high content of viable lactic acid bacteria. In an embodiment of the present invention the fermented product comprises one or more lactic acid bacterial strain(s) in a total amount in the range of 10⁵-10¹² CFU per gram of the fermented product, such as in the range of 10⁶-10¹² CFU per gram, e.g. in the range of 10²-10¹¹ CFU per gram, such as in the range of 10⁸-10¹¹ CFU per gram, e.g. in the range of 10⁸-10¹⁰ CFU per gram.

The fermentation process may preferably be a homofermentative fermentation process or a substantially homofermentative fermentation process.

In an embodiment of the present invention, the fermented product may comprise a lactic acid concentration above 1% (w/w) lactic acid relative to the fermented product, such as at least 1.5% (w/w) lactic acid relative to the fermented product, such as at least 2% (w/w) lactic acid relative to the fermented product, e.g. at least 3% (w/w) lactic acid relative to the fermented product, such as at least 4% (w/w) lactic acid relative to the fermented product, e.g. at least 5% (w/w) lactic acid relative to the fermented product, such as at least 6% (w/w) lactic acid relative to the fermented product, e.g. at least 7% (w/w) lactic acid relative to the fermented product, such as at least 8% (w/w) lactic acid relative to the fermented product, e.g. at least 9% (w/w) lactic acid relative to the fermented product, such as at least 10% (w/w) lactic acid relative to the fermented product, such as in the range of 1.1-10% (w/w) lactic acid relative to the fermented product, such as in the range of 2.5-7.5% (w/w) lactic acid relative to the fermented product, such as in the range of 5-6% (w/w) lactic acid relative to the fermented product.

In the context of the present invention, the term “substantially homofermentative” relates to an insignificant development of acetic acid during the homofermentative fermentation.

In an embodiment of the present invention the fermented product may comprise an acetic acid concentration in the range of 0.01-1% (w/w) acetic acid relative to the fermented product, such as in the range of 0.1-0.9% (w/w) acetic acid relative to the fermented product, such as in the range of 0.5-0.8% (w/w) acetic acid relative to the fermented product

The fourth parameter which may be found to be particular important for controlling the development of a metabolite during a fermentation process may be the fermentation process or the parameters of the fermentation process.

In an embodiment of the present invention the process involves an at-line or in-line determination of the at least 2 predetermined metabolites or a periodically sampling of fermented product to be analysed for the 2 predetermined metabolites.

In a further embodiment of the present invention the fermentation mixture may be allowed to ferment until the at least 2 predetermined metabolites are provided in desired amounts. In extended fermentations additional fermenting organisms may be added to the fermentation mixture to ensure high survival rate of the fermenting organisms in the fermented product.

In order to avoid too high temperature of the fermentation mixture, the fermentation mixture may be adjusted to a humidity in the range of 30-50% (w/w) humidity before the fermentation mixture is allowed to ferment in step (v), such as in the range of 35-45% (w/w) humidity, e.g. in the range of 38-42% (w/w) humidity, such as about 40% (w/w) humidity.

Air and/or oxygen may preferably be removed or substantially removed from the fermentation reactor before the fermentation mixture is allowed to ferment in step (v).

In the present context the term “substantially removed” relates to a presence of air and/or oxygen in the fermentation mixture of less than 5% (v/v) relative to the total volume of the fermentation mixture; such as less than 3% (v/v) relative to the total volume of the fermentation mixture; e.g. less than 1% (v/v) relative to the total volume of the fermentation mixture; such as less than 0.5% (v/v) relative to the total volume of the fermentation mixture; e.g. less than 0.1% (v/v) relative to the total volume of the fermentation mixture.

In an embodiment of the present invention the process may be an anaerobic fermentation process.

In a further embodiment of the present invention, the fermentation mixture may be maintained at a temperature of the fermentation mixture during the fermentation process which is below 50° C., such as below 45° C.; e.g. below 40° C., in order to maintain as many fermenting organisms alive. Preferably, the fermentation process may be performed at a temperature in the range of 15-40° C., such as 25-35° C., such as 30-40° C., such as 15-20° C. or such as 40-45° C.

The fermentation mixture may be allowed to ferment until the pH of the fermentation mixture reach a pH of pH 5.5 or below, such as a pH 5.0 or below; e.g. a pH 4.5 or below, such as a pH 4.3 or below, e.g. a pH 4.2 or below, such as a pH 4.1 or below, e.g. a pH 4.0 or below.

As the at least one plant material and/or the at least one seaweed material is not sterilized during the entire process, the growth of naturally occurring microorganisms (wanted microorganisms or unwanted microorganisms) are suppressed by the reduced ph and/or by the anaerobic fermentation conditions.

The fermentation mixture may preferably be allowed to ferment for at least 5 days, such as at least 7 days, e.g. for at least 10 days, such as for at least 12 days, e.g. for at least 15 days, such as for at least 17 days, e.g. for at least 20 days, such as for at least 23 days.

In an embodiment of the present invention, the fermented product obtained may be dried providing a dried fermented product. An example of methods for drying the fermented product according to the present invention reference is made to WO 2013/029632 which is hereby incorporate by reference.

Preferably, the dried fermented product may have a moisture content in the range of 4-12% (w/w), such as in the range of 5-10% (w/w), e.g. in the range of 6-8% (w/w).

When the fermented product comprises the combination of at least one fermented plant material and at least one fermented seaweed material, the fermentation of the at least one plant material and the fermentation of the at least one seaweed material may be performed separately or jointly. Preferably, the fermentation of the at least one plant material and the fermentation of the at least one seaweed material may be performed jointly.

The fermented product according to the present invention may preferably comprises a fibrous material. Preferably the fermented product comprises the fibrous material originating from the plant material and/or the seaweed material.

In an embodiment of the present invention, the fermented product comprises more than 5 g fibrous material per kg dried fermented product, such as more than 10 g fibrous material per kg dried fermented product, e.g. more than 15 g fibrous material per kg dried fermented product, such as more than 20 g fibrous material per kg dried fermented product, e.g. more than 25 g fibrous material per kg dried fermented product, such as more than 50 g fibrous material per kg dried fermented product, e.g. more than 75 g fibrous material per kg dried fermented product, such as more than 100 g fibrous material per kg dried fermented product, e.g. more than 150 g fibrous material per kg dried fermented product, such as more than 200 g fibrous material per kg dried fermented product, e.g. more than 250 g fibrous material per kg dried fermented product, such as more than 300 g fibrous material per kg dried fermented product.

The fermented product according to the present invention may preferably comprises a starch material. Preferably the fermented product comprises the starch material originating from the plant material and/or the seaweed material.

In an embodiment of the present invention, wherein the composition may comprise more than 5 g starch material per kg dried composition, such as more than 10 g starch material per kg dried composition, e.g. more than 15 g starch material per kg dried composition, such as more than 20 g starch material per kg dried composition, e.g. more than 25 g starch material per kg dried composition, such as more than 50 g starch material per kg dried composition, e.g. more than 75 g starch material per kg dried composition, such as more than 100 g starch material per kg dried composition, e.g. more than 150 g starch material per kg dried composition, such as more than 200 g starch material per kg dried composition, e.g. more than 250 g starch material per kg dried composition, such as more than 300 g starch material per kg dried composition.

At least one plant material and/or the at least one seaweed material may have an average maximum diameter of 5 cm, such as an average maximum diameter of 4 cm such as an average maximum diameter of 3 cm, such as an average maximum diameter of 2 cm, such as an average maximum diameter of 1 cm, such as an average diameter in the range 25 μm to 5 cm, such as 0.1 mm to 5 cm, such as an average diameter in the range of 0.5 mm to 5 cm, such as an average diameter in the range 0.5 mm to 2 cm.

The fermented product may be a dried composition comprises a range of 30-70% (w/w); such as a range of 40-60% (w/w); e.g. about 50% (w/w) of the dry composition has a particle size below 0.5 mm and a range of 30-70% (w/w); such as a range of 40-60% (w/w); e.g. about 50% (w/w) of the dry composition has a particle size above 0.5 mm.

In a further embodiment of the present invention the dried fermented product comprises at least 2, preferably at least 3, even more preferably at least 4 of the following criteria:
a) 1-10% (w/w), such as about 5% (w/w), of the dry fermented product has a particle size above 1.0 mm;
b) 45-55% (w/w), such as about 50% (w/w), of the dry fermented product has a particle size between 0.5-1.0 mm;
c) 30-40% (w/w), such as about 50% (w/w), of the dry fermented product has a particle size between 0.25-0.5 mm; and/or
d) 5-15% (w/w), such as about 10% (w/w), of the dry fermented product has a particle size below 0.25 mm.

In the present context the term “about” relates to a variation on the stated amount of 10% or less, such as 5% or less, e.g. 1% or less.

The selection of the various particle sizes may be determined by sieving as known to the skilled person.

The fermented seaweed material and/or the fermented plant material, may preferably not be subjected to sterilisation in order to maintain the native metabolic nature of the materials.

A preferred embodiment of the present invention relates to a fermented product comprising:
(a) at least one plant material and/or at least one seaweed material;
(b) at least one fermenting organism;
(c) at least 2 metabolites, wherein the at least 2 metabolites are selected from an amino acid; a fatty acid; a bioactive phenol; a vitamin; an acid; a purine compound; a carbohydrate compound; a flavonoid compound; or a bacterial biomarker.

The fermented product may further comprises a fibrous compound; preferably the fibrous compound originates from the at least one plant material and/or at least one seaweed material.

Preferably, the fibrous compound when originating from the plant material has an average diameter of 5 mm or less, e.g. an average diameter of 3 mm or less, such as an average diameter of 2 mm or less, such as an average diameter of 1 mm or less, such as an average diameter in the range 25 μm to 3 mm, such as 0.1 mm to 2.5 mm, such as an average diameter in the range of 0.5 mm to 2.25 mm, such as an average diameter in the range 1.0 mm to 2 mm.

Preferably, the fibrous compound when originating from the seaweed material has an average diameter of 2 mm or less, such as an average diameter of 1.5 mm or less, such as an average diameter of 1 mm or less, such as an average diameter in the range 25 μm to 2 mm, such as 0.1 mm to 1.5 mm, such as an average diameter in the range of 0.5 mm to 1.25 mm, such as an average diameter in the range 0.75 mm to 1 mm.

In an embodiment of the present invention the plant material may be selected from the Brassicaceae family, preferably selected from at least one of a Brassica genus; sun flower; palm; soya, field beans, lupins; or a combination hereof. Preferably, the at least one Brassica genus may be selected from one or more species such as Brassica napus; Brassica oleracea; Brassica campestris; Brassica nigra; Sinapis alba (Brassica alba); Brassica juncea; Brassica rapa or a mixture hereof. Preferably, the at least one Brassica genus is selected from the group consisting of: including rape, rapeseed, canola, cabbage, broccoli, cauliflower, kale, Brussels sprouts, collard greens, savoy, kohlrabi, gai Ian, white mustard, Indian mustard, Chinese mustard, and black mustard seed powder.

In a preferred embodiment of the present invention the at least one plant material may be Brassica nigra; Sinapis alba (Brassica alba); Brassica juncea; white mustard, Indian mustard, Chinese mustard, and/or black mustard seed powder.

When the at least one plant material may be Brassica nigra; Sinapis alba (Brassica alba);

Brassica juncea; white mustard, Indian mustard, Chinese mustard, and/or black mustard seed powder, the at least 2 metabolites may preferably comprise at least one amino acid selected from the group consisting of lysine, methionine, phenylalanine, tyrosine, asparagine, threonine, proline, leucine, isoleusine, and valine; such as at least 2 amino acids selected from said group; e.g. at least 3 amino acids selected from said group; such as at least 4 amino acids selected from said group; e.g. at least 5 amino acids selected from said group; such as at least 6 amino acids selected from said group; e.g. at least 7 amino acids selected from said group; such as at least 8 amino acids selected from said group; e.g. at least 9 amino acids selected from said group; such as at least 10 amino acids selected from said group.

When the at least one plant material may be Brassica nigra; Sinapis alba (Brassica alba); Brassica juncea; white mustard, Indian mustard, Chinese mustard, and/or black mustard seed powder, the at least 2 metabolites comprise at least one acid, preferable at least one organic acid, preferably at least one aromatic organic acid, Preferably, at least one aromatic organic acid selected from benzoic acid, 4-hydroxyphenylacetic acid and/or sinapinic acid; most preferably the aromatic acid may be benzoic acid.

In an embodiment of the present invention the at least one fermenting organism may be a substantially viable fermenting organism.

In the present context the term “substantially viable” relates to live fermenting organisms in a total amount in the range of 10⁵-10¹² CFU per gram of the fermented product, such as in the range of 10⁶-10¹² CFU per gram, e.g. in the range of 10⁷-10¹¹ CFU per gram, such as in the range of 10⁸-10¹¹ CFU per gram, e.g. in the range of 10⁸-10¹⁶ CFU per gram.

In an embodiment of the present invention the fermented product comprises a protein content in the range of 10-60% (w/w), such as in the range of 15-50% (w/w), e.g. in the range of 20-40% (w/w), such as in the range of 30-35% (w/w).

Preferably, at least 50% of the proteins available in the fermented product are digestible protein, such as at least 60%, e.g. at least 70%, such as at least 80%, e.g. at least 90% such as at least 95%.

One of the metabolites that may get increased attention is benzoic acid.

Benzoic acid may be used in modern animal production as it is believed to improve animal development as well as antimicrobial activity and the use of benzoic acid in animal production may reduce the environmental impact. In improved animal development, benzoic acid has shown an improvement on weight gain and better feed conversion ratio

A preferred embodiment of the present invention relates to a process for providing a fermented product rich in benzoic acid, said method comprises the steps of:
(i) Selecting at least one plant material selected from white mustard, Indian mustard, Chinese mustard, and/or black mustard seed powder.
(ii) Mixing the at least one plant material selected from white mustard, Indian mustard, Chinese mustard, and/or black mustard seed powder with at least one fermenting organism in a fermentation reactor providing a fermentation mixture;
(iii) Allowing the fermentation mixture to ferment under fermentation conditions favouring the development of benzoic acid providing the fermented product rich in benzoic acid.

In an embodiment of the present invention the process for providing a fermented product rich in benzoic acid comprises at least 2 predetermined metabolites.

In a further embodiment of the present invention the benzoic acid is one of the at least 2 predetermined metabolites.

In the context of the present invention, the term “comprising”, which may be synonymous with the terms “including”, “containing” or “characterized by”, relates to an inclusive or open-ended listing of features and does not exclude additional, unrecited features or method steps. The term “comprising” leaves the claim open for the inclusion of unspecified ingredients even in major amounts.

In the context of the present invention, the term “consisting essentially of”, relates to a limitation of the scope of a claim to the specified features or steps and those features or steps, not mentioned and that do not materially affect the basic and novel characteristic(s) of the claimed invention.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Fermenting Various Plant Materials and Seaweed Materials and Combinations Hereof

32 different products comprising plant materials and/or seaweed material was prepared and analysed for the content of various metabolites.

The 32 different fermented products comprise:
1. Saccharina (2013), Ascophyllum; rape (BGG1700)
2. Saccharina, Ascophyllum; rape (BGG1702)
3. Camelina;
4. Camelina, Ascophyllum;
5. Camelina, Ascophyllum, Saccharina;
6. Rape;
7. Rape—0 day;
8. Rape—1 day;
9. Rape—2 day;
10. Rape—3 day;
11. Rape—4 day;
12. Rape, 10% Seaweed;
13. Rape, 10% Seaweed;
14. Rape, Seaweed
15. Soya—0 day;
16. Soya—1 day;
17. Soya—2 day;
18. Soya—3 day;
19. Soya—4 day;
20. Soya and protease—1 day;
21. Soya and protease—3 day;
22. Soya without protease—1 day;
23. Soya without protease—3 day;
24. Mustard;
25. Mustard, Ascophyllum;
26. Mustard, Ascophyllum, Saccharina;
27. Mustard, Ascophyllum, Saccharina;
28. Ascophyllum;
29. Saccharina;
30. FP1
31. FP2
32. FP3

Fermentation mixtures comprising plant material (rape, soya and/or mustard) were fermented using a lactic acid bacteria (Pediococcus acidilactici DSM 16243, Pediococcus pentosaceus DSM 12834 and Lactobacillus plantarum DSM 12837) blend for 11 days unless stated otherwise;

Fermentation mixtures comprising seaweed material (brown algae) were fermented using a lactic acid bacteria (Pediococcus acidilactici DSM 16243, Pediococcus pentosaceus DSM 12834 and Lactobacillus plantarum DSM 12837) blend for 18 days;

Fermentation mixtures comprising a combination of seaweed and plant material (rape, soya and/or mustard) were fermented using a blend of lactic acid bacteria (Pediococcus acidilactici DSM 16243, Pediococcus pentosaceus DSM 12834 and Lactobacillus plantarum DSM 12837) for a first phase where seaweed (brown algae) was fermented for 7 days followed by a second phase where the plant material is added and the fermentation of the combined product was continued for additionally 11 days.

Each fermentation procedure is performed as a solid stage fermentation and each fermentation mixture are compressed in a fermentation reactor which is closed airtightly, leaving no or substantially no air/oxygen pockets.

When the fermentation process has ended the fermented product is dried in a spin flash dryer to a moisture content of about 10% (w/w).

The fermentation of seaweed (brown algae), plant material and the combination of seaweed (brown algae) and plant material is a controlled process using lactic acid bacteria and not the natural microorganisms from the plant material or from the seaweed material. The seaweed material is pre-fermented for 7 days in containers with inoculum of lactic acid bacteria (Pediococcus acidilactici DSM 16243, Pediococcus pentosaceus DSM 12834 and Lactobacillus plantarum DSM 12837). Plant material (in this case cold-pressed) are mixed in the fermented seaweed material together with a second batch of lactic acid bacteria and fermentation is continued for 11 days and dried down to 10% moisture in a sensitive process securing survival of the lactic acid bacteria to 10⁶-10⁸ CFU/g.

Sample Extraction

Homogenization and metabolite extraction is performed in one step using the following procedure: One gram of sample is homogenized in cold methanol using an Ultra-turrax. From the supernatant 1.5 mL is transferred to a glass vial and dried under nitrogen flow. Samples are reconstituted in 1 mL milliQ water prior to analysis using GC-MS analysis (Gas Chromatography—Mass Spectrometry analysis) and HILIC-analysis (Hydrophilic interaction chromatography analysis).

Results:

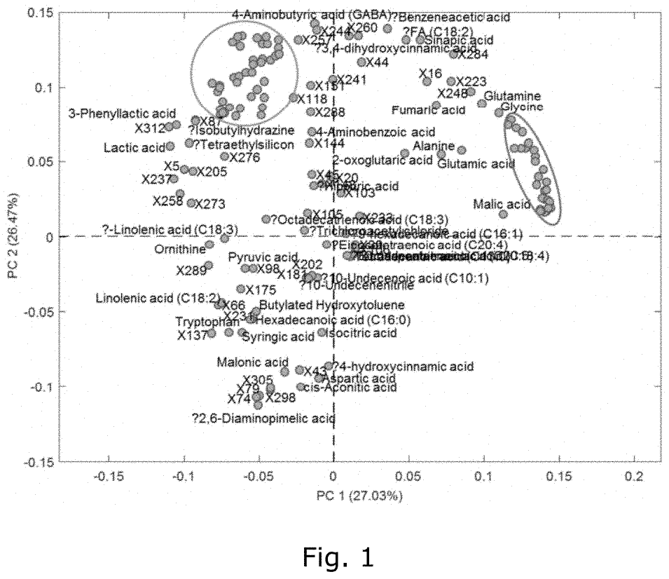

The results of the GC-MS analysis are summarised in FIG. 1 showing the development of a range of different metabolites where the fermentation processes are controlled by changing different parameters of the process like the plant material, the seaweed material and the process conditions.

FIG. 1 provides a fingerprint of a fermented product provided by fermenting a fermentation mixture comprising seaweed (brown algae) which is fermented using a blend of lactic acid bacteria (Pediococcus acidilactici DSM 16243, Pediococcus pentosaceus DSM 12834 and Lactobacillus plantarum DSM 12837) for 7 days followed by a second phase where a plant material (comprising rape and mustard) is added together with additional lactic acid bacteria (a blend of Pediococcus acidilactici DSM 16243, Pediococcus pentosaceus DSM 12834 and Lactobacillus plantarum DSM 12837) and the fermentation of the combined product was continued for additionally 11 days.

The fingerprint provided in FIG. 1 may be used as a guidance as to quality check of further fingerprint analyses provide for later productions.

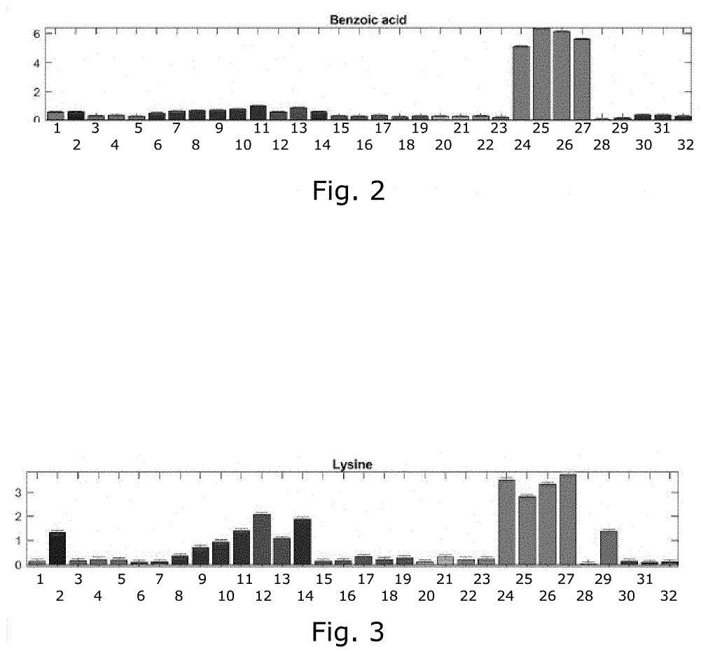

FIG. 2 shows the development of the metabolite, benzoic acid, during various fermentation conditions, as indicated above in the 32 different fermentation products.

Benzoic acid is an organic acid which is widely used in animal feed industry, due to its various advantageous effects, like antimicrobial effect, and antiseptic activity.

Interestingly, benzoic acid show to develop significantly when fermenting mustard plant materials. This significant development only show to appear when mustard are present. No benzoic acid is formed in the absence of mustard. Hence, rape, seaweed or soy does not lead to the formation of benzoic acid.

FIG. 3 shows the development of the metabolite, lysine, during various fermentation conditions, as indicated above in the 32 different fermentation products.

Lysine is an amino acid. The human body cannot synthesize lysine, so it is essential in e.g. humans poultry, swine, fish, crustaceans and dairy cows. This means that lysine must be obtained from the diet and is called an essential amino acid. Due to its importance in several biological processes, a lack of lysine can lead to several disease states including defective connective tissues, impaired fatty acid metabolism, anaemia, and systemic protein-energy deficiency.

FIG. 3 shows that by increasing the fermentation time of rape (see columns 7-11) an increased level of lysine is formed. The same phenomenon does not show when fermenting soy under similar conditions (see columns 15-19) where no increase in lysine concentration is observed. Furthermore, it is shown that mustard produces during the fermentation high amounts of lysine too.

Thus, in conclusion it is demonstrated that by controlling various parameters of the fermentation process it is possible to ensure an uniform fermentation product and to ensure fermentation products having the desired constituents.

### REFERENCES

WO 2013/029632

1. A process for controlling the development of at least 2 predetermined metabolites in a fermented product comprising at least one plant material and/or at least one seaweed material, wherein the process comprising the steps of:
(i) Determining the at least 2 predetermined metabolites to be developed in the fermented product;
(ii) Based on the predetermined metabolites determined in step (i) at least one plant material and/or the at least one seaweed material is selected;
(iii) Based on the predetermined metabolites determined in step (i) at least one fermenting organism is selected;
(iv) Mixing the at least one plant material and/or the at least one seaweed material selected in step (ii) with the at least one fermenting organism selected in step (iii) in a fermentation reactor providing a fermentation mixture;
(v) Allowing the fermentation mixture to ferment under fermentation conditions favouring the development of the at least 2 predetermined metabolites;
whereby the fermented product is provided.

2. The process according to claim 1, wherein the at least one seaweed material is a unicellular alga or a multicellular macroalgae.

3. The process according to claim 1, wherein the at least one plant material is Brassica nigra; Sinapis alba (Brassica alba); Brassica juncea; Brassica rapa or a mixture hereof.

4. The process according to claim 1, wherein the at least 2 metabolites are selected from an amino acid; a fatty acid; a bioactive phenol; a vitamin; an acid; a purine compound; a carbohydrate compound; a flavonoid compound; or a bacterial biomarker.

5. The process according to claim 4, wherein the acid is benzoic acid, or derivatives thereof.

6. The process according to claim 1, wherein the at least one fermenting organism is one or more health-enhancing microorganism.

7. The process according to claim 1, wherein a library of metabolites is identified in the fermented product and wherein the library of metabolites of the fermented product and/or the fermentation mixture is compared to the library of metabolites of a similar previous fermented product.

8. A fermented product comprising:
(a) at least one plant material and/or at least one seaweed material;
(b) at least one fermenting organism;
(c) at least 2 metabolites, wherein the at least 2 metabolites are selected from an amino acid; a fatty acid; a bioactive phenol; a vitamin; an acid; a purine compound; a carbohydrate compound; a flavonoid compound; or a bacterial biomarker.

9. The fermented product according to claim 8, wherein the fermented product further comprises a fibrous compound; wherein the fibrous compound originates from the at least one plant material and/or at least one seaweed material, and wherein the at least one plant material is Brassica nigra; Sinapis alba (Brassica alba); Brassica juncea; white mustard, Indian mustard, Chinese mustard, and/or black mustard seed powder

10. The fermented product according to claim 8, wherein the at least 2 metabolites comprise at least one acid.

11. The fermented product according to claim 1, wherein the multicellular macroalgae may be selected from brown macroalgae, and/or red macroalgae.

12. The fermended product according to claim 1, wherein the brown macroalgae may be selected from the group consisting of one or more of kelps, Saccharina latissimi (Laminaria saccharina), Laminaria digitate, Ascophyllum nodosum, Laminaria hyperborean, and a mixture thereof.

13. The fermented product according to claim 1, wherein the at least one fermenting organism is one or more health-enhancing microorganism in the form of one or more lactic acid bacteria.

14. The fermented product according to claim 10, wherein the at least one acid comprises at least one organic acid.

15. The fermented product according to claim 14, wherein the at least one organic acid comprises at least one aromatic organic acid.

16. The fermented product according to claim 15, wherein the at least one aromatic organic acid comprises benzoic acid.

17. The fermented product according to claim 10, wherein the at least 2 metabolites comprise lysine.

18. The fermented product according to claim 10, wherein the at least 2 metabolites comprise benzoic acid, lysine, and lactic acid.
